(19) United States
(12) Patent Application Publication
(10) Pub. No.: US 2018/0195920 A1
(43) Pub. Date: Jul. 12, 2018

(54) PRESSURE SENSING DEVICE, PRESSURE SENSING SYSTEM AND PRESSURE SWITCH USING SAME

(71) Applicant: SOLID YEAR CO., LTD., New Taipei City (TW)

(72) Inventors: Yen-Rong KUAN, Taipei City (TW); Chao-Yang HOU, New Taipei City (TW)

(21) Appl. No.: 15/531,631

(22) PCT Filed: May 12, 2016
(86) PCT No.: PCT/CN2016/081838

§ 371 Date: May 30, 2017

(51) Int. Cl. G01L 7/04; A61B 5/11; A61B 5/113; A61B 5/00; A61B 5/103

(52) U.S. Cl. CPC G01L 7/04; A61B 5/1102; A61B 5/113; A61B 2562/0252; A61B 5/6891; A61B 5/1036; A61B 2562/0247; A61B 5/6892

(57) ABSTRACT

A pressure sensing device includes an elastic tube defming a chamber and an opening in one or each of two opposite ends thereof in communication with the chamber, and a pressure sensor component sealed in the opening in one or each end of the elastic tube for detecting a change in air pressure in the chamber when the elastic tube is compressed by an external pressure. The pressure sensing device can be used under a waterproof or dustproof environment, or embedded in a bed mattress or seat cushion and incorporated with a signal processor for detecting changes in the biophysical condition of the user, or embedded in a floor mat and incorporated with light and/or sound generating device for reminding your home or store that someone is coming in or going out. Thus, the pressure sensing device has the characteristics of simple structure, practical applicability and wide application range.

### BACKGROUND OF THE INVENTION

### 1. Field of the Invention

The present invention relates to pressure sensing technology and more particularly, to a pressure sensing device for multiple applications, a pressure sensing system using the pressure sensing device, and a pressure switch using the pressure sensing device.

### 2. Description of the Related Art

A sensor is a device capable of converting measured physical or chemical quantity of an object into a usable output signal according to certain rules. Sensors are widely used in the fields of automotive, medical, industrial measurement and automatic control.

In terms of health care, the traditional way is to use a wearable sensing device for sensing the patient's biophysical state, and to link the wearable sensing device to computer for monitoring whether the patient has an abnormal situation. However, it needs professional medical personnel to control the operation. Further, this method is not suitable for use in patients who require long-term disease tracking. Further, the wearable sensing device can fall off due to the patient's pain and uncomfortability or purposeless involuntary movements. This will inevitably affect the sensing result. Thus, the conventional wearable sensing device is not convenient in use.

Further, in terms of automatic control technique, such as motion sensing device, as long as someone passes the sensing range, it will automatically turn on the lights or give off sound to alert the home, achieving home security. However, this kind of sensing devices are usually installed outdoors, they are vulnerable to external environmental factors (such as rain or dust), causing a structural damage or short circuit. Thus, in actual application, this kind of sensing devices cannot provide a satisfactory warning effect.

### SUMMARY OF THE INVENTION

The present invention has been accomplished under the circumstances in view. It is the main object of the present invention to provide a pressure sensing device, which has the characteristics of simple structure, practical applicability and wide application range.

To achieve this and other objects of the present invention a pressure sensing device comprises an elastic tube, which comprises a chamber defined therein and an opening located in one or each of two opposite ends thereof in communication with the chamber, and a pressure sensor component sealed in the opening in one or each opening of the elastic tube for detecting a change in air pressure in the chamber and sending out a sensing signal when the elastic tube is compressed by an external pressure. Subject to the airtight protective effect of the elastic tube, the pressure sensing device can be used under a waterproof or dustproof environment. Further, because the pressure sensor component is sealed in the elastic tube, it will not be affected or damaged by external environmental factors.

It is another object of the present invention to provide a pressure sensing system, which is practical for detecting changes in the biophysical condition of the user.

To achieve this and other objects of the present invention a pressure sensing system is created by using the aforesaid pressure sensing device to incorporate with a signal processing device. The signal processing device is adapted for receiving a sensing signal from the pressure sensor component and converting it into a biophysical signal. In application, the pressure sensing device is embedded in a bed mattress or a seat cushion of a wheelchair or any other chair for detecting changes in the biophysical condition of the user (such as heartbeat, breathing or body movements). Once an abnormal condition occurs, the signal processing device sends out a warning signal in the shortest time, so that the medical personnel can carry out emergency medical treatments immediately.

It is still another object of the present invention to provide a pressure switch, which is practical for home protection application, or for for home or store access monitoring to remind your home or store that someone is coming in or going out.

To achieve this and other objects of the present invention a pressure switch is created by using the aforesaid pressure sensing device to incorporate with a controller and a load device. The controller is electrically coupled with the pressure sensor component, and adapted for receiving a sensing signal from the pressure sensor component and sending out a control signal according to the received sensing signal. The load device is electrically connected to the controller, and adapted for receiving the control signal from the controller and turning on or off according to the recessed control signal.

Thus, the pressure sensing device can be embedded in a floor mat in front of the front entrance door of a house or store. When someone is walking out of or into the house or store over the floor mat, the controller will control the load device to close or open the front entrance door. Further, a light and/or sound generating device can be simultaneously drive to emit light and/or sound, reminding your home or store that someone is coming in or going out, and thus, making it possible to achieve enhanced home or store protection.

Other advantages and features of the present invention will be fully understood by reference to the following specification in conjunction with the accompanying drawings, in which like reference signs denote like components of structure.

### BRIEF DESCRIPTION OF THE DRAWINGS

FIG. 1 is an exploded view of a pressure sensing device in accordance with the present invention.

FIG. 2 is a schematic sectional view of the pressure sensing device in accordance with the present invention.

FIG. 3 is a sectional view of an alternate form of the pressure sensing device in accordance with the present invention.

FIG. 4 is a sectional view of another alternate foirn of the pressure sensing device in accordance with the present invention.

FIG. 5 is a block diagram of a pressure switch in accordance with the present invention.

FIG. 6 is a schematic applied view of the present invention, illustrating the pressure sensing device embedded in a floor mat.

FIG. 7 is another schematic applied view of the present invention, illustrating the pressure sensing device embedded in a bed mattress.

FIG. 8 is still another schematic applied view of the present invention, illustrating a different mounting arrangement of the pressure sensing device in a bed mattress.

FIG. 9 us a schematic sectional view illustrating the pressure sensing device embedded in a bed mattress.

FIG. 10 is still another schematic applied view of the present invention, illustrating the pressure sensing device embedded in a seat cushion of a wheelchair.

FIG. 11 is a block diagram of a pressure sensing system in accordance with the present invention.

### DETAILED DESCRIPTION OF THE INVENTION

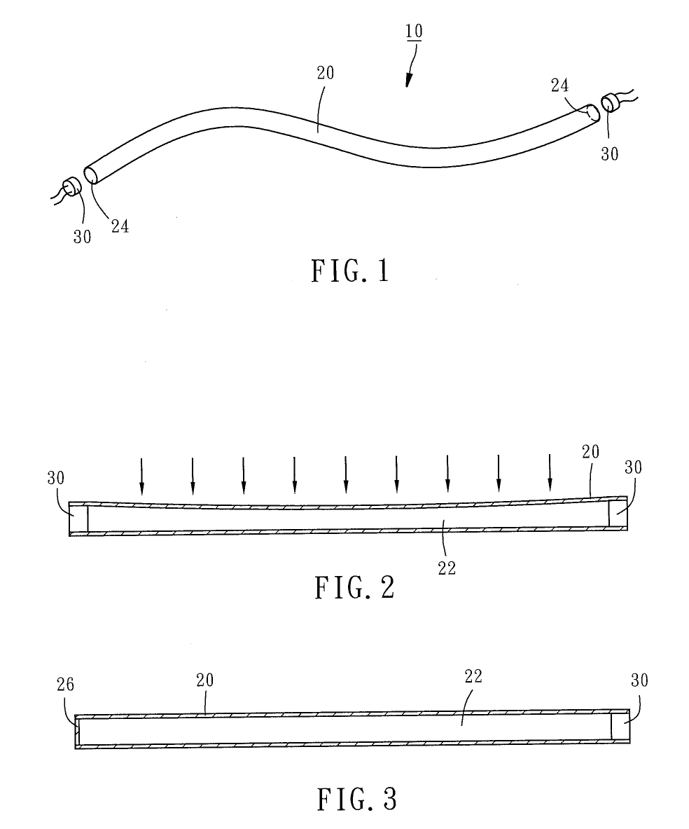

Referring to FIGS. 1 and 2, a pressure sensing device 10 in accordance with the present invention is shown. The pressure sensing device 10 comprises an elastic tube 20 and two pressure sensor components 30.

The elastic tube 20 is made from silicon rubber, rubber, or any other similar elastic material, defining an opening 24 in each of two opposite ends thereof and a chamber 22 in communication between the two openings 24.

The pressure sensor components 30 can be piezoelectric, piezoresistive or capacitive pressure sensor components for microphone application. These two pressure sensor components 30 are respectively press-fitted into the two openings 24 of the elastic tube 20 to seal the chamber 22 of the elastic tube 20.

As illustrated in FIG. 2, when an external pressure is acted on the elastic tube 20, the internal air in the chamber 22 is compressed, causing a change in air pressure. At this time, the pressure sensor components 30 sense the change in air pressure and output a sensing signal. Once the external pressure disappears, the elastic tube 20 immediately returns to its original shape.

Further, in an alternate form of the pressure sensing device 10 as shown in

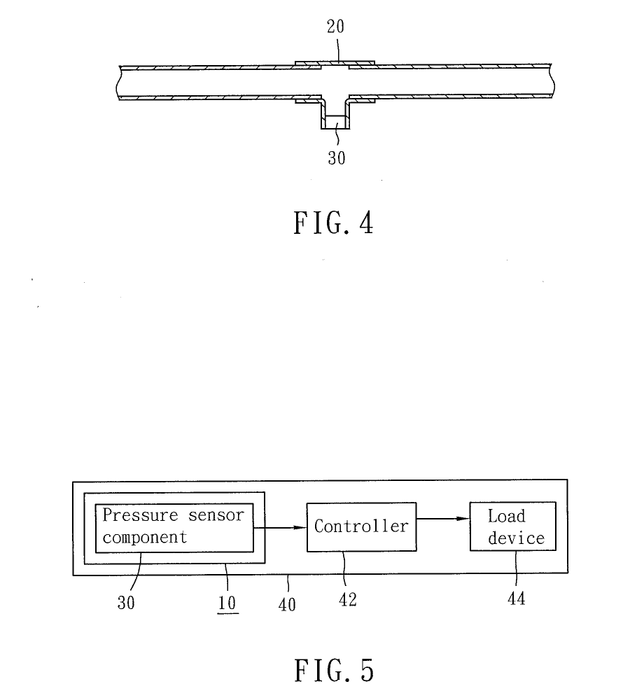

FIG. 3, the elastic tube 20 has only one opening 24 that is located in one end the elastic tube 20 and sealed with one pressure sensor component 30; the other end of the elastic tube 20 is blocked by an end wall 26; the two opposite ends of the chamber 22 of the elastic tube 20 are blocked by the end wall 26 and the pressure sensor component 30. In another alternate form of the pressure sensing device 10 as shown in FIG. 4, the elastic tube 20 is a three-way tube, having its one end sealed with one pressure sensor component 30 and its other two ends configured for the connection of other different designs of elastic tubes to extend the length.

In actual application, the pressure sensing device 10 can be used in different situations.

At first, subject to the airtight protective effect of the elastic tube 20, the pressure sensing device 10 can be used under a waterproof or dustproof environment. Further, because the pressure sensor components 30 are sealed in the elastic tube 20, they will not be affected or damaged by external environmental factors. Further, the pressure sensing device 10 can be incorporated with a controller 42 and a load device 44 to create a pressure switch 40, as shown in FIG. 5. When the controller 42 receives a sensing signal from the pressure sensor components 30, it will send out a control signal to the load device 44 according to the sensing signal, driving the load device 44 to turn on or off.

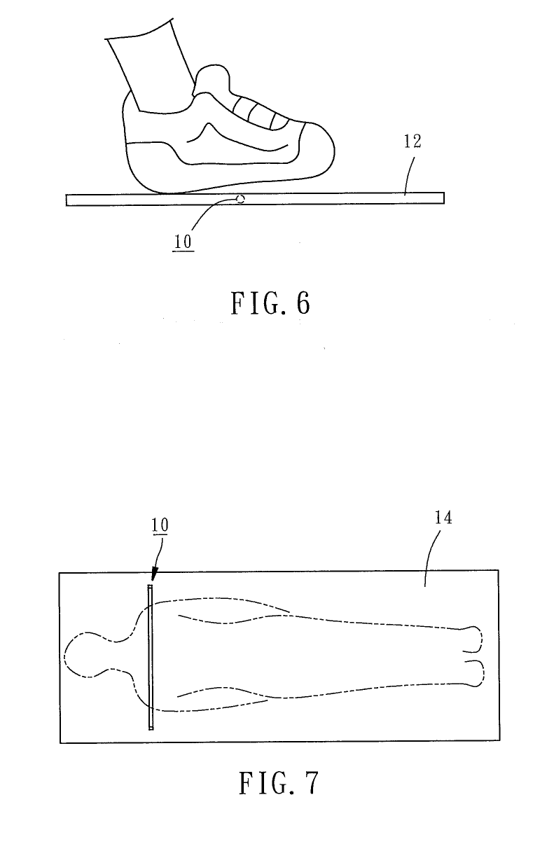

Further, as shown in FIG. 6, the pressure sensing device 10 can be embedded in a floor mat 12 in front of the front entrance door of a house or store. When someone is walking out of or into the house or store over the floor mat 12, the elastic tube 20 will be compressed by the pressure from the person walking over the floor mat 12. When the elastic tube 20 is compressed, the pressure sensor components 30 are forced to provide a sensing signal to the controller 42, causing the controller 42 to drive the load device 44 to turn on or off, i.e., to close or open the front entrance door. When the load device 44 is driven to turn on or off, a light and/or sound generating device can be simultaneously drive to emit light and/or sound, reminding your home or store that someone is coming in or going out, and thus, making it possible to achieve enhanced home or store protection.

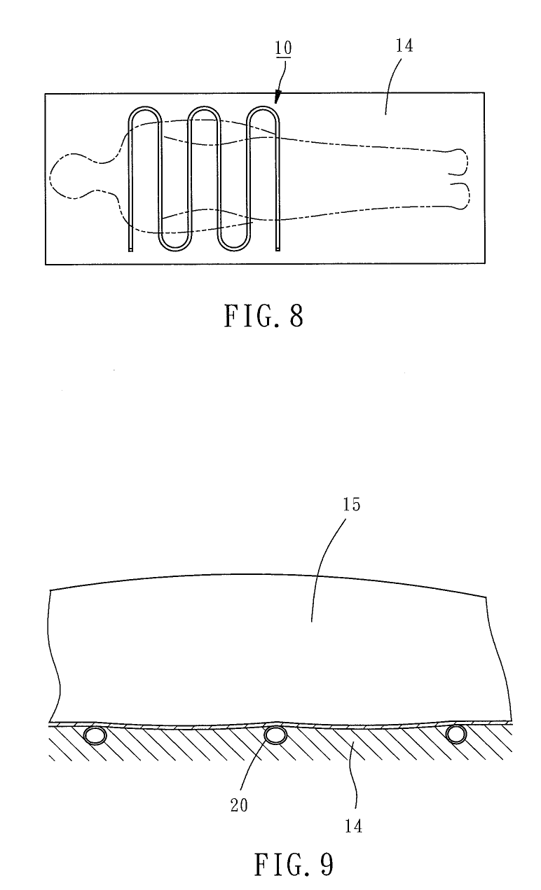

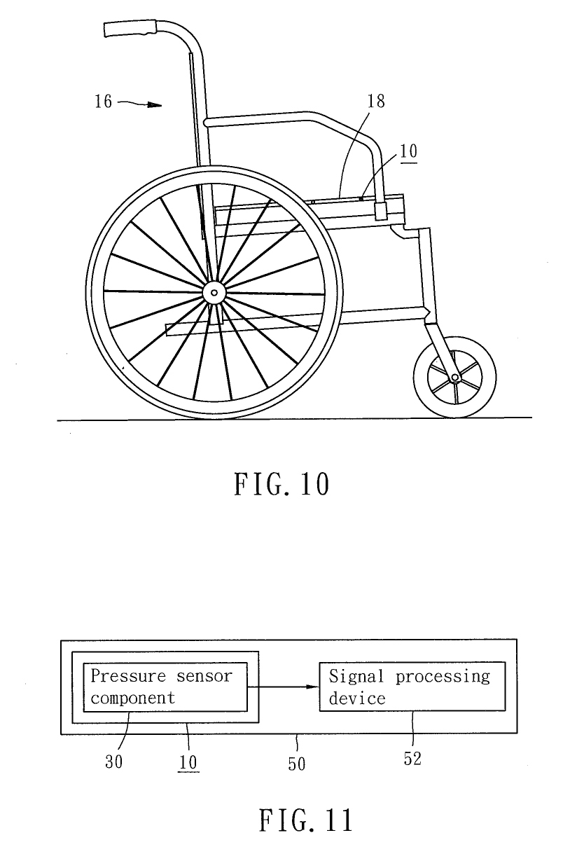

Referring to FIGS. 7-9, the pressure sensing device 10 can be embedded in a bed mattress 14 for sickbed. The embedded position of the pressure sensing device 10 in the bed mattress 14 is adjustable subject to its length. If the length of the pressure sensing device 10 is short, the optimal embedded position is to let the pressure sensing device 10 face toward the chest of the patient. If the length of the pressure sensing device 10 is sufficient, the optimal embedded position of the pressure sensing device 10 in the bed mattress 14 is to let the pressure sensing device 10 face toward the patient's torso. Thus, when the patient is lying on the bed mattress 14, the patient's heart beats, breathing or any body movement will give different pressures to the elastic tube 20. At this time, a signal processing device 52 can be incorporated with the pressure sensing device 10 to create a pressure sensing system 50, as shown in FIG. 11. When the signal processing device 52 receives a sensing signal from the pressure sensor components 30 of the pressure sensing device 10, the signal processing device 52 immediately processes this sensing signal and converts it into a biophysical signal. Once an abnormal situation occurs in the patient, for example, no heartbeat, stopping breathing or fell off the bed, the signal processing device 52 displays an a biophysical signal abnormality and sends out a warning signal in the shortest time, so that the medical personnel can carry out emergency medical treatments immediately. Under the same principle, the pressure sensing system 50 can be used to detect the quality of sleep of an ordinary person, or embedded in a seat cushion 18 of a wheelchair 16, as shown in FIG. 10, to detect the body movements of the patient sitting in the wheelchair 16, achieving biophysical monitoring.

In conclusion, by means of utilizing the sealing effect and material properties of the elastic tube 20 to match with the sensing function of the pressure sensor components 30, the pressure sensing device 10 can be used under specific environments, and can simultaneously be used to monitor the biophysical conditions of a patient, or can even be used for home or store access monitoring to remind your home or store that someone is coming in or going out. Thus, the pressure sensing device 10 of the present invention has the characteristics of simple structure, practical applicability and wide application range.

What is claimed is:

1. A pressure sensing device, comprising:
at least one elastic tube comprising a chamber defined therein and at least one opening located in at least one of two opposite ends thereof in communication with said chamber; and
at least one pressure sensor component sealed in said at least one opening of said elastic tube for detecting a change in air pressure in said chamber when said at least one elastic tube is compressed by an external pressure.

2. The pressure sensing device as claimed in claim 1, wherein said at least one elastic tube comprises only one single said opening located in the one end thereof, and an end wall sealing the other end thereof; said chamber is defined in said at least one elastic tube between said end wall and the one single said pressure sensor component that is sealed in the one single said opening in one end of said at least one elastic tube.

3. The pressure sensing device as claimed in claim 1, wherein said at least one elastic tube comprises two said openings respectively located the two opposite ends thereof; the number of said at least one pressure sensor component is 2, and these two said pressure sensor components are respectively sealed in the two said openings in the two opposite ends of said at least one elastic tube; said chamber is defined in said at least one elastic tube between the two said pressure sensor components.

4. A pressure sensing system, comprising:
a pressure sensing device as claimed in claim 1; and
a signal processing device electrically coupled with said at least one pressure sensor component and adapted for receiving and processing a sensing signal produced by said at least one pressure sensor component.

5. The pressure sensing system as claimed in claim 4, wherein said at least one elastic tube of said pressure sensing device comprises only one single said opening located in the one end thereof, and an end wall sealing the other end thereof; said chamber is defined in said at least one elastic tube between said end wall and the one single said pressure sensor component that is sealed in the one single said opening in one end of said at least one elastic tube.

6. The pressure sensing system as claimed in claim 4, wherein said at least one elastic tube of said pressure sensing device comprises two said openings respectively located the two opposite ends thereof; the number of said at least one pressure sensor component is 2, and these two said pressure sensor components are respectively sealed in the two said openings in the two opposite ends of said at least one elastic tube; said chamber is defined in said at least one elastic tube between the two said pressure sensor components.

7. A pressure switch, comprising:
a pressure sensing device as claimed in claim 1;
a controller electrically coupled with said at least one pressure sensor component and adapted for receiving a sensing signal produced by said at least one pressure sensor component and sending out a control signal according to the received said sensing signal; and
a load device electrically connected to said controller and adapted for receiving said control signal from said controller and turning on or off according to the recessed said control signal.

8. The pressure switch as claimed in claim 7, wherein said at least one elastic tube of said pressure sensing device comprises only one single said opening located in the one end thereof, and an end wall sealing the other end thereof; said chamber is defined in said elastic tube between said end wall and the one single said pressure sensor component that is sealed in the one single said opening in one end of said elastic tube.

9. The pressure switch as claimed in claim 7, wherein said at least one elastic tube of said pressure sensing device comprises two said openings respectively located the two opposite ends thereof; the number of said at least one pressure sensor component is 2, and these two said pressure sensor components are respectively sealed in the two said openings in the two opposite ends of said at least one elastic tube; said chamber is defined in said at least one elastic tube between the two said pressure sensor components.
